(19) United States
(12) Patent Application Publication
(10) Pub. No.: US 2025/0222061 A1
(43) Pub. Date: Jul. 10, 2025

(54) MULTI-VITAMIN FOR ADJUVANT TREATMENT OF AIDS, AND PREPARATION METHOD AND APPLICATION METHOD THEREOF

(71) Applicant: Changde Jizhi Biological Technology Co., Ltd, Changde (CN)

(72) Inventor: Xinqiao HE, Changde (CN)

(21) Appl. No.: 18/405,886

(22) Filed: Jan. 5, 2024

(51) Int. Cl. A61K 36/8984; A61K 31/205; A61K 33/10; A61K 36/068; A61K 36/25; A61K 36/286; A61K 36/36; A61K 36/65; A61K 36/8945; A61K 38/00; A61P 31/18

(52) U.S. Cl. CPC A61K 36/8984; A61K 31/205; A61K 33/10; A61K 36/068; A61K 36/25; A61K 36/286; A61K 36/36; A61K 36/65; A61K 36/8945; A61K 38/00; A61P 31/18

(57) ABSTRACT

A multi-vitamin for adjuvant treatment of AIDS, and a preparation method and application thereof are provided. The multi-vitamin has good therapeutic effect, can quickly balance nutrition, supplement energy, and enhance immunity, such that by virtue of an immune system and a self-healing function of a user of the multi-vitamin, bacteria and viruses can be killed, damaged cells can be repaired, and cell regeneration capacity and metabolism are improved.

### TECHNICAL FIELD

The disclosure relates to the field of biomedical technologies, particularly to a multi-vitamin for adjuvant treatment of acquire immune deficienc syndrome (AIDS), and a preparation method and application method thereof.

### BACKGROUND

A pathogen of AIDS is a human immunodeficiency virus (HIV). At present, AIDS has become an important public health problem that seriously threatens public health in China. According to the joint evaluation by Chinese Center for Disease Control and Prevention (China CDC), Joint United Nations Program on HIV/AIDS (UNAIDS), and World Health Organization (WHO), by the end of the year of 2018, there were about 1.25 million AIDS patients in China, with an estimated 80,000 new infections per year, an infection rate of about 9.0 per 10,000 in the whole population, and a total of about 262,000 deaths reported. In the past 10 years, great progress has been made in the treatment of AIDS, and a total of six categories of antiretroviral drugs, including of more than 30 kinds of antiretroviral drugs have been obtained. The goal of highly active antiretroviral therapy (HAART) is to inhibit HIV replication to the maximum extent, and thus to enhance immune function, however, treatment is ineffective if a patient is not able to insist on treatment. Non-compliance or partial compliance with a treatment plan for HAART can result in failure of viral inhibition or even exacerbation of disease or death as the virus develops resistance to antiretroviral drugs. Long-term adherence to the antiretroviral drugs is a challenge for patients with AIDS, and compliance will decrease over time with increased adverse drug reactions. As a result of improved disease control by HAART, life span of HIV-infected people will be gradually prolonged, however, the HIV-infected people are more likely to suffer from other chronic diseases, such as diabetes and heart disease. As more and more age-related complications appear, the compliance of patients with complex treatment solutions is reduced, and the possibility of hospitalization and adverse drug events is increased.

In general, the current research on AIDS treatment covers: new oral long-acting antiretroviral drugs and other types of long-acting antiretroviral drugs, simplified treatment strategies, immunization-based treatment, HIV reservoir, preventive and therapeutic vaccines and other aspects. However, how to translate science into practice and policy is still an arduous task for AIDS treatment. In the absence of effective vaccines, the widespread use of effective antiretroviral drugs for treatment and prevention is still the key to halting the global AIDS epidemic. It is hoped that future research on AIDS treatment will cover more areas, including pre-exposure prevention of HIV, prevention of mother-to-child transmission, and research on comorbidities including hepatitis, so as to bring better prevention and treatment to the HIV-infected people, prolong their lives and improve their quality of life.

Because HIV can destroy the human immune system and has extremely strong variability, the existing drugs can hardly play a role in inhibiting HIV. Even if the drugs can work temporarily, drug resistance will occur after a period of time, and a therapeutic effect will gradually weaken or even lose.

Therefore, how to make the life span of AIDS patients close to that of normal people through treatment under the premise of effectiveness and safety is a technical problem to be solved in the medical field.

### SUMMARY

The main objective of the disclosure is to provide a multi-vitamin for adjuvant treatment of AIDS, and a preparation method of the multi-vitamin for adjuvant treatment of AIDS, and an application method of the multi-vitamin for adjuvant treatment of AIDS. The multi-vitamin disclosed by the disclosure has a good treatment effect, and can quickly balance nutrition, supplement energy, and enhance immunity, such that by virtue of an immune system and a self-healing function of a user of the multi-vitamin, bacteria and viruses can be killed, damaged cells can be repaired, and cell regeneration capacity and metabolism are improved.

Compared with the related art, the technical solutions of the disclosure are as follows.

An embodiment of the disclosure provides a multi-vitamin for adjuvant treatment of AIDS. The multi-vitamin is made from raw materials including: 4-6 parts by weight of shiitake peptide, 5-15 parts by weight of Cordyceps sinensis, 5-15 parts by weight of Dendrobium officinale, 10-20 parts by weight of prepared rhubarb, 10-20 parts by weight of Radix paeoniae alba, 5-10 parts by weight of safflower, 10-20 parts by weight of Rhizoma dioscoreae, 5-10 parts by weight of Angelica sinensis, 2-4 parts by weight of L-carnitine, and 3-6 parts by weight of sodium bicarbonate.

In an embodiment, the raw materials include: 4 parts by weight of the shiitake peptide, parts by weight of the Cordyceps sinensis, 5 parts by weight of the Dendrobium officinale, 10 parts by weight of the prepared rhubarb, 10 parts by weight of the Radix paeoniae alba, 5 parts by weight of the safflower, 10 parts by weight of the Rhizoma dioscoreae, 5 parts by weight of the Angelica sinensis, 2 parts by weight of the L-carnitine, and 3 parts by weight of the sodium bicarbonate.

In an embodiment, the raw materials include: 5 parts by weight of the shiitake peptide, parts by weight of the Cordyceps sinensis, 10 parts by weight of the Dendrobium officinale, 15 parts by weight of the prepared rhubarb, 15 parts by weight of the Radix paeoniae alba, 7 parts by weight of the safflower, 15 parts by weight of the Rhizoma dioscoreae, 7 parts by weight of the Angelica sinensis, 3 parts by weight of the L-carnitine, and 4 parts by weight of the sodium bicarbonate.

In an embodiment, the raw materials include: 6 parts by weight of the shiitake peptide, parts by weight of the Cordyceps sinensis, 15 parts by weight of the Dendrobium officinale, 20 parts by weight of the prepared rhubarb, 20 parts by weight of the Radix paeoniae alba, 10 parts by weight of the safflower, 20 parts by weight of the Rhizoma dioscoreae, 10 parts by weight of the Angelica sinensis, 4 parts by weight of the L-carnitine, and 6 parts by weight of the sodium bicarbonate.

In an embodiment, an preparation method of the multi-vitamin is provided, and includes the following steps: step 1, adding water into the Cordyceps sinensis and the Dendrobium officinale with a weight of the water being 20-30 times of a weight of the Cordyceps sinensis, decocting for 150-200 minutes (min), and filtering by using an 800-mesh filter screen to obtain a mixed nutrient solution; putting the mixed nutrient solution into a fermentation tank, and adding mucor and brown sugar into the mixed nutrient solution for fermenting for 80-100 days to obtain a fermentation product, a weight of the mucor being 0.25%-0.65% of a weight of the mixed nutrient solution, a weight of the brown sugar being 5%-8% of the weight of the mixed nutrient solution; taking out the fermentation product and adding anhydrous ethanol into the fermentation product, a weight of the anhydrous ethanol being 30%-50% of the weight of the mixed nutrient solution, standing for 12-24 hours, and then filtering to obtain a filtrate; and concentrating and drying the filtrate to obtain a standby material; step 2, adding water into the prepared rhubarb, the Radix paeoniae alba, the safflower, the Rhizoma dioscoreae, and Angelica sinensis, decocting for 60-120 min, and filtering to obtain for a first decoction filtrate and a residue; adding water into the residue and then decocting for twice each for 60-90 min, and filtering to obtain second and third decoction filtrates; mixing the first, second, and third filtrates to obtain a mixed filtrate; and concentrating and drying the mixed filtrate to obtain a prepared rhubarb extract; and step 3, uniformly mixing the standby material obtained in the step 1 with the prepared rhubarb extract obtained in the step 2 to obtain a mixture; and adding the shiitake peptide, the L-carnitine and the sodium bicarbonate into the mixture, and then uniformly mixing to thereby obtain the multi-vitamin.

An embodiment of the disclosure provides an application method of the multi-vitamin, and the application method includes: applying the multi-vitamin to prepare an AIDS adjuvant treatment drug.

An embodiment of the disclosure provides a traditional Chinese medicine composition for adjuvant treatment of AIDS, which includes the multi-vitamin and disinfection enzyme.

In an embodiment, a weight ratio of the multi-vitamin:the disinfection enzyme is (6-10):(2-6).

The disclosure has at least the following beneficial effects.

In the disclosure, on the one hand, the multi-vitamin is extracted from multiple rare traditional Chinese medicines, plants and foods and contains multiple vitamins, trace elements, rich protein and minerals, can quickly balance nutrition, supplement energy, and enhance immunity, such that by virtue of an immune system and a self-healing function of a user of the multi-vitamin, bacteria and viruses can be killed, damaged cells can be repaired, and cell regeneration capacity and metabolism are improved. On the other hand, by using small molecular water vapor body therapy, nano-scale active lytic enzyme extracted from protein after repeated fermentation can be added to small molecular water. Therefore, steam body therapy for patients can expand capillaries and expel free radicals and killed bacteria and viruses in bodies of the patients. By eating the multi-vitamin, the patients can supplement and balance nutrition and quickly restore immune function thereof.

### DETAILED DESCRIPTION OF EMBODIMENTS

In order to make the objectives, technical solutions and advantages of the disclosure more clearly, the disclosure will be further described in detail in combination with specific embodiments. It should be understood that these descriptions are merely exemplary and are not intended to limit the scope of protection of the disclosure. In addition, in the following description, descriptions of well-known structures and technologies are omitted to avoid unnecessarily confusing the concepts of the disclosure.

### Embodiment 1

The embodiment 1 provides a multi-vitamin for adjuvant treatment of AIDS. The multi-vitamin is made from raw materials including: 4 parts by weight of shiitake peptide, 5 parts by weight of Cordyceps sinensis, 5 parts by weight of Dendrobium officinale, 10 parts by weight of prepared rhubarb, 10 parts by weight of Radix paeoniae alba, 5 parts by weight of safflower, 10 parts of Rhizoma dioscoreae, 5 parts by weight of Angelica sinensis, 2 parts by weight of L-carnitine, and 3 parts by weight of sodium bicarbonate.

The embodiment 1 further provides a preparation method of the multi-vitamin, and the preparation method includes the following steps:
step 1, adding water into the Cordyceps sinensis and the Dendrobium officinale with a weight of the water being 20 times of a weight of the Cordyceps sinensis, decocting for 150 minutes (min), and filtering by using an 800-mesh filter screen to obtain a mixed nutrient solution; putting the mixed nutrient solution into a fermentation tank, and adding mucor and brown sugar into the mixed nutrient solution for fermenting for 80 days to obtain a fermentation product, a weight of the mucor being 0.25% of a weight of the mixed nutrient solution, a weight of the brown sugar being 5% of the weight of the mixed nutrient solution; taking out the fermentation product and adding anhydrous ethanol into the fermentation product, a weight of the anhydrous ethanol being 30% of the weight of the mixed nutrient solution, standing for 12 hours, and then filtering to obtain a filtrate; and concentrating and drying the filtrate to obtain a standby material;
step 2, adding water into the prepared rhubarb, the Radix paeoniae alba, the safflower, the Rhizoma dioscoreae, and Angelica sinensis, decocting for 60 min, and filtering to obtain for a first decoction filtrate and a residue; adding water into the residue and then decocting for twice each for 60 min and filtering to obtain second and third decoction filtrates, mixing the first, second, and third filtrates to obtain a mixed filtrate; and concentrating and drying the mixed filtrate to obtain an extract; and step 3, uniformly mixing the standby material obtained in the step 1 with the extract obtained in the step 2 to obtain a mixture; and adding the shiitake peptide, the L-carnitine and the sodium bicarbonate into the mixture, and then uniformly mixing to obtain the multi-vitamin.

### Embodiment 2

The embodiment 2 provides a multi-vitamin for adjuvant treatment of AIDS. The multi-vitamin is made from raw materials including: 6 parts by weight of shiitake peptide, 15 parts by weight of Cordyceps sinensis, 15 parts by weight of Dendrobium officinale, 20 parts by weight of prepared rhubarb, 20 parts by weight of Radix paeoniae alba, 10 parts by weight of safflower, 20 parts of Rhizoma dioscoreae, 10 parts by weight of Angelica sinensis, 4 parts by weight of L-carnitine, and 6 parts by weight of sodium bicarbonate.

The embodiment 2 further provides a preparation method of the multi-vitamin, and the preparation method includes the following steps:
step 1, adding water into the Cordyceps sinensis and the Dendrobium officinale with a weight of the water being 30 times of a weight of the Cordyceps sinensis, decocting for 200 min, and filtering by using an 800-mesh filter screen to obtain a mixed nutrient solution; putting the mixed nutrient solution into a fermentation tank, and adding mucor and brown sugar into the mixed nutrient solution for fermenting for 100 days to obtain a fermentation product, a weight of the mucor being 0.65% of a weight of the mixed nutrient solution, a weight of the brown sugar being 8% of the weight of the mixed nutrient solution; taking out the fermentation product and adding anhydrous ethanol into the fermentation product, a weight of the anhydrous ethanol being 50% of the weight of the mixed nutrient solution, standing for 24 hours, and then filtering to obtain a filtrate; and concentrating and drying the filtrate to obtain a standby material;
step 2, adding water into the prepared rhubarb, the Radix paeoniae alba, the safflower, the Rhizoma dioscoreae, and Angelica sinensis, decocting for 120 min, and filtering to obtain for a first decoction filtrate and a residue; adding water into the residue and then decocting for twice each for 90 min and filtering to obtain second and third decoction filtrates, mixing the first, second, and third filtrates to obtain a mixed filtrate; and concentrating and drying the mixed filtrate to obtain an extract; and step 3, uniformly mixing the standby material obtained in the step 1 with the extract obtained in the step 2 to obtain a mixture; and adding the shiitake peptide, the L-carnitine and the sodium bicarbonate into the mixture, and then uniformly mixing to obtain the multi-vitamin.

### Embodiment 3

The embodiment 3 provides a multi-vitamin for adjuvant treatment of AIDS. The multi-vitamin is made from raw materials including: 5 parts by weight of shiitake peptide, 8 parts by weight of Cordyceps sinensis, 8 parts by weight of Dendrobium officinale, 13 parts by weight of prepared rhubarb, 11 parts by weight of Radix paeoniae alba, 5 parts by weight of safflower, 13 parts of Rhizoma dioscoreae, 6 parts by weight of Angelica sinensis, 3 parts by weight of L-carnitine, and 4 parts by weight of sodium bicarbonate.

The embodiment 3 further provides a preparation method of the multi-vitamin, and the preparation method includes the following steps:
step 1, adding water into the Cordyceps sinensis and the Dendrobium officinale with a weight of the water being 20 times of a weight of the Cordyceps sinensis, decocting for 150 minutes (min), and filtering by using an 800-mesh filter screen to obtain a mixed nutrient solution; putting the mixed nutrient solution into a fermentation tank, and adding mucor and brown sugar into the mixed nutrient solution for fermenting for 80 days to obtain a fermentation product, a weight of the mucor being 0.25% of a weight of the mixed nutrient solution, a weight of the brown sugar being 5% of the weight of the mixed nutrient solution; taking out the fermentation product and adding anhydrous ethanol into the fermentation product, a weight of the anhydrous ethanol being 30% of the weight of the mixed nutrient solution, standing for 12 hours, and then filtering to obtain a filtrate; and concentrating and drying the filtrate to obtain a standby material;
step 2, adding water into the prepared rhubarb, the Radix paeoniae alba, the safflower, the Rhizoma dioscoreae, and Angelica sinensis, decocting for 60 min, and filtering to obtain for a first decoction filtrate and a residue; adding water into the residue and then decocting for twice each for 60 min and filtering to obtain second and third decoction filtrates, mixing the first, second, and third filtrates to obtain a mixed filtrate; and concentrating and drying the mixed filtrate to obtain an extract; and
step 3, uniformly mixing the standby material obtained in the step 1 with the extract obtained in the step 2 to obtain a mixture; and adding the shiitake peptide, the L-carnitine and the sodium bicarbonate into the mixture, and then uniformly mixing to obtain the multi-vitamin.

### Embodiment 4

The embodiment 4 provides a multi-vitamin for adjuvant treatment of AIDS. The multi-vitamin is made from raw materials including: 4 parts by weight of shiitake peptide, 7 parts by weight of Cordyceps sinensis, 9 parts by weight of Dendrobium officinale, 11 parts by weight of prepared rhubarb, 13 parts by weight of Radix paeoniae alba, 7 parts by weight of safflower, 10 parts of Rhizoma dioscoreae, 10 parts by weight of Angelica sinensis, 3 parts by weight of L-carnitine, and 3 parts by weight of sodium bicarbonate.

The embodiment 4 further provides a preparation method of the multi-vitamin, and the preparation method includes the following steps:
step 1, adding water into the Cordyceps sinensis and the Dendrobium officinale with a weight of the water being 20 times of a weight of the Cordyceps sinensis, decocting for 150 minutes (min), and filtering by using an 800-mesh filter screen to obtain a mixed nutrient solution; putting the mixed nutrient solution into a fermentation tank, and adding mucor and brown sugar into the mixed nutrient solution for fermenting for 80 days to obtain a fermentation product, a weight of the mucor being 0.25% of a weight of the mixed nutrient solution, a weight of the brown sugar being 5% of the weight of the mixed nutrient solution; taking out the fermentation product and adding anhydrous ethanol into the fermentation product, a weight of the anhydrous ethanol being 30% of the weight of the mixed nutrient solution, standing for 12 hours, and then filtering to obtain a filtrate; and concentrating and drying the filtrate to obtain a standby material;
step 2, adding water into the prepared rhubarb, the Radix paeoniae alba, the safflower, the Rhizoma dioscoreae, and Angelica sinensis, decocting for 60 min, and filtering to obtain for a first decoction filtrate and a residue; adding water into the residue and then decocting for twice each for 60 min and filtering to obtain second and third decoction filtrates, mixing the first, second, and third filtrates to obtain a mixed filtrate; and concentrating and drying the mixed filtrate to obtain an extract; and
step 3, uniformly mixing the standby material obtained in the step 1 with the extract obtained in the step 2 to obtain a mixture; and adding the shiitake peptide, the L-carnitine and the sodium bicarbonate into the mixture, and then uniformly mixing to obtain the multi-vitamin.

### Embodiment 5

The embodiment 5 provides a multi-vitamin for adjuvant treatment of AIDS. The multi-vitamin is made from raw materials including: 6 parts by weight of shiitake peptide, 12 parts by weight of Cordyceps sinensis, 13 parts by weight of Dendrobium officinale, 16 parts by weight of prepared rhubarb, 15 parts by weight of Radix paeoniae alba, 5 parts by weight of safflower, 15 parts of Rhizoma dioscoreae, 6 parts by weight of Angelica sinensis, 4 parts by weight of L-carnitine, and 3 parts by weight of sodium bicarbonate.

The embodiment 5 further provides a preparation method of the multi-vitamin, and the preparation method includes the following steps:
step 1, adding water into the Cordyceps sinensis and the Dendrobium officinale with a weight of the water being 20 times of a weight of the Cordyceps sinensis, decocting for 150 minutes (min), and filtering by using an 800-mesh filter screen to obtain a mixed nutrient solution; putting the mixed nutrient solution into a fermentation tank, and adding mucor and brown sugar into the mixed nutrient solution for fermenting for 80 days to obtain a fermentation product, a weight of the mucor being 0.25% of a weight of the mixed nutrient solution, a weight of the brown sugar being 5% of the weight of the mixed nutrient solution; taking out the fermentation product and adding anhydrous ethanol into the fermentation product, a weight of the anhydrous ethanol being 30% of the weight of the mixed nutrient solution, standing for 12 hours, and then filtering to obtain a filtrate; and concentrating and drying the filtrate to obtain a standby material;
step 2, adding water into the prepared rhubarb, the Radix paeoniae alba, the safflower, the Rhizoma dioscoreae, and Angelica sinensis, decocting for 60 min, and filtering to obtain for a first decoction filtrate and a residue; adding water into the residue and then decocting for twice each for 60 min and filtering to obtain second and third decoction filtrates, mixing the first, second, and third filtrates to obtain a mixed filtrate; and concentrating and drying the mixed filtrate to obtain an extract; and
step 3, uniformly mixing the standby material obtained in the step 1 with the extract obtained in the step 2 to obtain a mixture; and adding the shiitake peptide, the L-carnitine and the sodium bicarbonate into the mixture, and then uniformly mixing to obtain the multi-vitamin.

### Embodiment 6

The embodiment 6 provides a multi-vitamin for adjuvant treatment of AIDS. The multi-vitamin is made from raw materials including: 4 parts by weight of shiitake peptide, 10 parts by weight of Cordyceps sinensis, 10 parts by weight of Dendrobium officinale, 18 parts by weight of prepared rhubarb, 18 parts by weight of Radix paeoniae alba, 10 parts by weight of safflower, 10 parts of Rhizoma dioscoreae, 5 parts by weight of Angelica sinensis, 3 parts by weight of L-carnitine, and 5 parts by weight of sodium bicarbonate.

The embodiment 6 further provides a preparation method of the multi-vitamin, and the preparation method includes the following steps:
step 1, adding water into the Cordyceps sinensis and the Dendrobium officinale with a weight of the water being 20 times of a weight of the Cordyceps sinensis, decocting for 150 minutes (min), and filtering by using an 800-mesh filter screen to obtain a mixed nutrient solution; putting the mixed nutrient solution into a fermentation tank, and adding mucor and brown sugar into the mixed nutrient solution for fermenting for 80 days to obtain a fermentation product, a weight of the mucor being 0.25% of a weight of the mixed nutrient solution, a weight of the brown sugar being 5% of the weight of the mixed nutrient solution; taking out the fermentation product and adding anhydrous ethanol into the fermentation product, a weight of the anhydrous ethanol being 30% of the weight of the mixed nutrient solution, standing for 12 hours, and then filtering to obtain a filtrate; and concentrating and drying the filtrate to obtain a standby material;
step 2, adding water into the prepared rhubarb, the Radix paeoniae alba, the safflower, the Rhizoma dioscoreae, and Angelica sinensis, decocting for 60 min, and filtering to obtain for a first decoction filtrate and a residue; adding water into the residue and then decocting for twice each for 60 min and filtering to obtain second and third decoction filtrates, mixing the first, second, and third filtrates to obtain a mixed filtrate; and concentrating and drying the mixed filtrate to obtain an extract; and
step 3, uniformly mixing the standby material obtained in the step 1 with the extract obtained in the step 2 to obtain a mixture; and adding the shiitake peptide, the L-carnitine and the sodium bicarbonate into the mixture, and then uniformly mixing to obtain the multi-vitamin.

### Comparative Example 1

The comparative example 1 differs from the embodiment 1 only in that the comparative example 1 does not contain Cordyceps sinensis, and other drug substances and a preparation method are the same as those in the embodiment 1.

### Comparative Example 2

The comparative example 2 differs from the embodiment 1 only in that the comparative example 2 does not contain Dendrobium officinale, and other drug substances and a preparation method are the same as those in the embodiment 1.

### Comparative Example 3

The comparative example 3 differs from the embodiment 1 only in that the comparative example 3 does not contain prepared rhubarb, and other drug substances and a preparation method are the same as those in the embodiment 1.

### Test Example 1

Effect on immunosuppressed mice is determined through the test example 1.

Test animals are healthy male mice of Kunming strain.

investigational drugs are lamivudine tablets produced by Anhui Biochem Bio-Pharmaceutical Co., Ltd, multi-vitamin obtained in the embodiments, and multi-vitamin obtained in the comparative examples 1-3.

A test group 1 (also referred to as lamivudine group) uses lamivudine tablets.

A test group 2 (also referred to as embodiment and lamivudine group) uses lamivudine tablets and a product obtained in the embodiment 1.

A test group 3 (also referred to as embodiment and lamivudine group) uses lamivudine tablets and a product obtained in the embodiment 2.

A test group 4 (also referred to as embodiment and lamivudine group) uses lamivudine tablets and a product obtained in the embodiment 3.

A test group 5 (also referred to as embodiment and lamivudine group) uses lamivudine tablets and a product obtained in the embodiment 4.

A test group 6 (also referred to as embodiment and lamivudine group) uses lamivudine tablets and a product obtained in the embodiment 5.

A test group 7 (also referred to as embodiment and lamivudine group) uses lamivudine tablets and a product obtained in the embodiment 6.

A test group 8 (also referred to as comparative example and lamivudine group) uses lamivudine tablets and a product obtained in the comparative example 1.

A test group 9 (also referred to as comparative example and lamivudine group) uses lamivudine tablets and a product obtained in the comparative example 2.

A test group 10 (also referred to as comparative example and lamivudine group) uses lamivudine tablets and a product obtained in the comparative example 3.

A Test method includes the following steps. Mice are randomly divided into a control group and test groups 1-10, and are weighed. Each of the control group and the test groups 1-10 is injected with cyclosporine A dissolved in 35% of ethanol at a dose of 25 milligrams per kilogram per day (mg/kg/d) through abdominal cavity once every other day for a total of 3 times. From the next day after the last administration of cyclosporin A, drugs are given to the test groups by gavage for seven consecutive days. Specifically, zidovudine is given to the test group 1 at a dose of 45 mg/kg by gavage once a day; zidovudine at a dose of 45 mg/kg and a product of a corresponding embodiment or comparative example at a dose of 1.5 grams per kilograms (g/kg) are given to the test group 2-10 by gavage once a day. From the next day after the last administration of cyclosporin A, normal saline is given to the control group by gavage, once a day, for seven consecutive days. After 8 h of fasting for solids and liquids, each of the control group and test groups 1-10 is narcotized with ether; and 2 milliliters (mL) of blood is extracted using a cardiac blood sampling method from an apical pulse site of each of the control group and test groups 1-10 using a syringe with a volume of 1 mL, immediately transferred into an ethylene diamine tetraacetic acid (EDTA) anticoagulant tube, and mixed fully and uniformly. A ratio of CD₄⁺ T cell subsets and CD₈⁺ T cell subsets is detected and analyzed according to an instruction of a kit. Each of the control group and test groups 1-10 are killed, and thymus and spleen of each of the control group and test groups 1-10 are harvested and weighed, a spleen mass index and a thymus mass index of each of the control group and test groups 1-10 are calculated by the following formula: an index (mg/g)=a mass of the thymus or the spleen (mg)/a body mass (g). Test results are presented in table 1 and table 2

TABLE 1
Effect of each of the control group and the test groups 1-10
on the spleen mass index and the thymus mass index in mice

| | Number of | Thymus mass index | Spleen mass index |
|---|---|---|---|
| Group | mice | (mg/g) | (mg/g) |
| Control group | 10 | 1.74 ± 0.22 | 3.66 ± 0.39 |
| Test group 1 | 10 | 2.28 ± 0.19* | 3.41 ± 0.43 |
| Test group 2 | 10 | 2.67 ± 0.46**# | 3.89 ± 0.62 |
| Test group 3 | 10 | 2.54 ± 0.41**# | 3.76 ± 0.65 |
| Test group 4 | 10 | 2.57 ± 0.36**# | 3.80 ± 0.52 |
| Test group 5 | 10 | 2.55 ± 0.34**# | 3.72 ± 0.61 |
| Test group 6 | 10 | 2.82 ± 0.58**## | 3.92 ± 0.79 |
| Test group 7 | 10 | 2.97 ± 0.53**## | 4.05 ± 0.71 |
| Test group 8 | 10 | 2.37 ± 0.34* | 3.53 ± 0.39 |
| Test group 9 | 10 | 2.26 ± 0.28* | 3.59 ± 0.44 |
| Test group 10 | 10 | 2.36 ± 0.32* | 3.47 ± 0.46 |

Note:
compared with the control group,
**P < 0.01, and
*P < 0.05; and compared with the test group 1,
##P < 0.01, and
#P < 0.05.

TABLE 2
Effects of each of the control group and the test groups 1-10 on the
ratio of CD₄⁺ T cell subsets and CD₈⁺ T cell subsets

| Group | Number of animals | CD₄⁺/CD₈⁺ |
|---|---|---|
| Control group | 10 | 1.36 ± 0.37 |
| Test group 1 | 10 | 1.72 ± 0.34* |
| Test group 2 | 10 | 2.52 ± 0.28**# |
| Test group 3 | 10 | 2.44 ± 0.19**# |
| Test group 4 | 10 | 2.47 ± 0.27**# |
| Test group 5 | 10 | 2.46 ± 0.23**# |
| Test group 6 | 10 | 2.77 ± 0.49**## |
| Test group 7 | 10 | 2.89 ± 0.40**## |
| Test group 8 | 10 | 1.84 ± 0.28* |
| Test group 9 | 10 | 1.92 ± 0.33* |
| Test group 10 | 10 | 1.79 ± 0.22* |

Note:
compared with the control group,
**P < 0.01, and
*P < 0.05; and compared with the test group 1,
##P < 0.01, and
#P < 0.05.

Conclusions of experiment are as follows. (1) Spleen is the largest lymphoid organ in animals, and its function is closely related to the immune function of animals. Thymus is one of the most important immune organs in animals, which mainly controls the differentiation and maturation of T cells. The integrity of a structure and a function of the thymus is closely related to the level of immunity of animals. From the data in the table 1, it can be seen that a thymus mass index and a spleen mass index of the lamivudine group, i.e., the test group 1, are improved, while a thymus mass index and a spleen mass index of each of the embodiment and lamivudine groups, i.e., the test groups 2-7, are further improved, and have significant or extremely significant differences compared with the lamivudine group. However, each of the comparative example and lamivudine groups, i.e., the test groups 8-10, has no statistical significance compared with the lamivudine group, which fully shows that the products of the disclosure can improve the immunity together with the lamivudine, further promote the efficacy of the lamivudine in treating AIDS, and has the function of adjuvant treatment of AIDS. (2) Acquired immunodeficiency syndrome is a disease caused by HIV infection, which seriously damages human immune system and thus poses a serious threat to patients' lives. With the progress of the disease of the patents, CD₄⁺ T cells are gradually reduced or even completely exhausted, and CD₄⁺/CD₈⁺ of an HIV-infected patient is significantly lower than that of a normal person. The data in the table 2 of the disclosure show that CD₄⁺/CD₈⁺ of the lamivudine group is improved, while CD₄⁺/CD₈⁺ of each of the embodiment and lamivudine groups, i.e., the test groups 2-7, is further improved, and has significant or extremely significant differences compared with the lamivudine group. However, each of the comparative example and lamivudine groups, i.e., the test groups 8-10, has no statistical significance compared with the lamivudine group, which fully shows that the products of the disclosure can improve the immunity together with the lamivudine, further promote the efficacy of the lamivudine in treating AIDS, and has the function of adjuvant treatment of AIDS.

The above is merely the exemplary embodiments of the disclosure, and it should be pointed out that a person skilled in the art can make several improvements and embellishments without departing from the principle of the disclosure, and these improvements and embellishments should also be regarded as falling into the scope of protection of the disclosure.

What is claimed is:

1. A multi-vitamin for adjuvant treatment of acquire immune deficienc syndrome (AIDS), wherein the multi-vitamin is made from raw materials comprising: 4-6 parts by weight of shiitake peptide, 5-15 parts by weight of Cordyceps sinensis, 5-15 parts by weight of dendrobium officinale, 10-20 parts by weight of prepared rhubarb, 10-20 parts by weight of Radix paeoniae alba, 5-10 parts by weight of safflower, 10-20 parts by weight of Rhizoma dioscoreae, 5-10 parts by weight of Angelica sinensis, 2-4 parts by weight of L-carnitine, and 3-6 parts by weight of sodium bicarbonate.

2. The multi-vitamin for adjuvant treatment of AIDS as claimed in claim 1, wherein the raw materials comprise: 4 parts by weight of the shiitake peptide, 5 parts by weight of the Cordyceps sinensis, 5 parts by weight of the Dendrobium officinale, 10 parts by weight of the prepared rhubarb, 10 parts by weight of the Radix paeoniae alba, 5 parts by weight of the safflower, 10 parts by weight of the Rhizoma dioscoreae, 5 parts by weight of the Angelica sinensis, 2 parts by weight of the L-carnitine, and 3 parts by weight of the sodium bicarbonate.

3. The multi-vitamin for adjuvant treatment of AIDS as claimed in claim 1, wherein the raw materials comprise: 5 parts by weight of the shiitake peptide, 10 parts by weight of the Cordyceps sinensis, 10 parts by weight of the Dendrobium officinale, 15 parts by weight of the prepared rhubarb, 15 parts by weight of the Radix paeoniae alba, 7 parts by weight of the safflower, 15 parts by weight of the Rhizoma dioscoreae, 7 parts by weight of the Angelica sinensis, 3 parts by weight of the L-carnitine, and 4 parts by weight of the sodium bicarbonate.

4. The multi-vitamin for adjuvant treatment of AIDS as claimed in claim 1, wherein the raw materials comprise: 6 parts by weight of the shiitake peptide, 15 parts by weight of the Cordyceps sinensis, 15 parts by weight of the Dendrobium officinale, 20 parts by weight of the prepared rhubarb, 20 parts by weight of the Radix paeoniae alba, 10 parts by weight of the safflower, 20 parts by weight of the Rhizoma dioscoreae, 10 parts by weight of the Angelica sinensis, 4 parts by weight of the L-carnitine, and 6 parts by weight of the sodium bicarbonate.

5. The multi-vitamin for adjuvant treatment of AIDS as claimed in claim 1, wherein the raw materials comprise: 5 parts by weight of the shiitake peptide, 8 parts by weight of the Cordyceps sinensis, 8 parts by weight of the Dendrobium officinale, 13 parts by weight of the prepared rhubarb, 11 parts by weight of the Radix paeoniae alba, 5 parts by weight of the safflower, 13 parts by weight of the Rhizoma dioscoreae, 6 parts by weight of the Angelica sinensis, 3 parts by weight of the L-carnitine, and 4 parts by weight of the sodium bicarbonate.

6. The multi-vitamin for adjuvant treatment of AIDS as claimed in claim 1, wherein the raw materials comprise: 4 parts by weight of the shiitake peptide, 7 parts by weight of the Cordyceps sinensis, 9 parts by weight of the Dendrobium officinale, 11 parts by weight of the prepared rhubarb, 13 parts by weight of the Radix paeoniae alba, 7 parts by weight of the safflower, 10 parts by weight of the Rhizoma dioscoreae, 10 parts by weight of the Angelica sinensis, 3 parts by weight of the L-carnitine, and 3 parts by weight of the sodium bicarbonate.

7. The multi-vitamin for adjuvant treatment of AIDS as claimed in claim 1, wherein the raw materials comprise: 6 parts by weight of the shiitake peptide, 12 parts by weight of the Cordyceps sinensis, 13 parts by weight of the Dendrobium officinale, 16 parts by weight of the prepared rhubarb, 15 parts by weight of the Radix paeoniae alba, 5 parts by weight of the safflower, 15 parts by weight of the Rhizoma dioscoreae, 6 parts by weight of the Angelica sinensis, 4 parts by weight of the L-carnitine, and 3 parts by weight of the sodium bicarbonate.

8. The multi-vitamin for adjuvant treatment of AIDS as claimed in claim 1, wherein the raw materials comprise: 4 parts by weight of the shiitake peptide, 10 parts by weight of the Cordyceps sinensis, 10 parts by weight of the Dendrobium officinale, 18 parts by weight of the prepared rhubarb, 18 parts by weight of the Radix paeoniae alba, 10 parts by weight of the safflower, 10 parts by weight of the Rhizoma dioscoreae, 5 parts by weight of the Angelica sinensis, 3 parts by weight of the L-carnitine, and 5 parts by weight of the sodium bicarbonate.

9. An preparation method of a multi-vitamin for adjuvant treatment of AIDS, wherein the multi-vitamin is made from raw materials comprising: 4-6 parts by weight of shiitake peptide, 5-15 parts by weight of Cordyceps sinensis, 5-15 parts by weight of Dendrobium officinale, 10-20 parts by weight of prepared rhubarb, 10-20 parts by weight of Radix paeoniae alba, 5-10 parts by weight of safflower, 10-20 parts by weight of Rhizoma dioscoreae, 5-10 parts by weight of Angelica sinensis, 2-4 parts by weight of L-carnitine, and 3-6 parts by weight of sodium bicarbonate;
wherein the preparation method of the multi-vitamin comprises the following steps:
step 1, adding water into the Cordyceps sinensis and the Dendrobium officinale with a weight of the water being 20-30 times of a weight of the Cordyceps sinensis to obtain a first mixture, decocting the first mixture for 150-200 minutes (min), and filtering by using an 800-mesh filter screen to obtain a mixed nutrient solution; putting the mixed nutrient solution into a fermentation tank, and adding mucor and brown sugar into the mixed nutrient solution for fermenting for a fermentation time of 80-100 days to obtain a fermentation product, a weight of the mucor being 0.25%-0.65% of a weight of the mixed nutrient solution, a weight of the brown sugar being 5%-8% of the weight of the mixed nutrient solution; taking out the fermentation product and adding anhydrous ethanol into the fermentation product, a weight of the anhydrous ethanol being 30%-50% of the weight of the mixed nutrient solution, standing for a standing time of 12-24 hours, and then filtering to obtain a filtrate; and concentrating and drying the filtrate to obtain a standby material;
step 2, adding water into the prepared rhubarb, the Radix paeoniae alba, the safflower, the Rhizoma dioscoreae, and Angelica sinensis to obtain a second mixture, decocting the second mixture for 60-120 min, and filtering to obtain for a first decoction filtrate and a residue; adding water into the residue to obtain a residue solution, and then decocting the residue solution for twice each for 60-90 min, and filtering to obtain second and third decoction filtrates; mixing the first, second, and third filtrates to obtain a mixed filtrate; and concentrating and drying the mixed filtrate to obtain a prepared rhubarb extract; and
step 3, uniformly mixing the standby material obtained in the step 1 with the prepared rhubarb extract obtained in the step 2 to obtain a mixture; and adding the shiitake peptide, the L-carnitine and the sodium bicarbonate into the mixture, and then uniformly mixing to thereby obtain the multi-vitamin.

10. The preparation method of a multi-vitamin for adjuvant treatment of AIDS as claim in claim 9, wherein the weight of the water added into the Cordyceps sinensis and the Dendrobium officinale is 20 times of the weight of the Cordyceps sinensis; a time for decocting the first mixture is 150 min; the fermentation time is 80 days; the weight of the mucor is 0.25% of the weight of the mixed nutrient solution; the weight of the brown sugar is 5% of the weight of the mixed nutrient solution; the weight of the anhydrous ethanol is 30% of the weight of the mixed nutrient solution; and the standing time is 12 hours;
wherein a time for decocting the second mixture is 60 min; and a time for each of the twice is 60 min.

11. The preparation method of a multi-vitamin for adjuvant treatment of AIDS as claim in claim 9, wherein the weight of the water added into the Cordyceps sinensis and the Dendrobium officinale is 30 times of the weight of the Cordyceps sinensis; a time for decocting the first mixture is 200 min; the fermentation time is 100 days; the weight of the mucor is 0.65% of the weight of the mixed nutrient solution; the weight of the brown sugar is 8% of the weight of the mixed nutrient solution; the weight of the anhydrous ethanol is 50% of the weight of the mixed nutrient solution; and the standing time is 24 hours;
wherein a time for decocting the second mixture is 120 min; and a time for each of the twice is 90 min.

12. An application method of the multi-vitamin as claimed in claim 1, comprising:
applying the multi-vitamin to prepare an AIDS adjuvant treatment drug.

13. A traditional Chinese medicine composition for adjuvant treatment of AIDS, comprising:
the multi-vitamin as claimed in claim 1; and
disinfection enzyme.

14. The traditional Chinese medicine composition as claimed in claim 13, wherein a weight ratio of the multi-vitamin:the disinfection enzyme is (6-10):(2-6).

15. A multi-vitamin for adjuvant treatment of AIDS, wherein the multi-vitamin is made from raw materials comprising: Cordyceps sinensis, Dendrobium officinale, mucor, brown sugar, anhydrous ethanol, prepared rhubarb, Radix paeoniae alba, safflower, Rhizoma dioscoreae, Angelica sinensis, shiitake peptide, L-carnitine, and sodium bicarbonate.

16. The multi-vitamin for adjuvant treatment of AIDS as claimed in claim 15, wherein a weight ratio of the shiitake peptide:the Cordyceps sinensis:the Dendrobium officinale:the prepared rhubarb:the Radix paeoniae alba:the safflower:the Rhizoma dioscoreae:the angelica sinensis:the L-carnitine:the sodium bicarbonate is 4-6:5-15:5-15:10-20:10-20:5-10:10-20:5-10:2-4:3-6.

17. The multi-vitamin for adjuvant treatment of AIDS as claimed in claim 15, wherein a weight ratio of the shiitake peptide:the Cordyceps sinensis:the Dendrobium officinale:the prepared rhubarb:the Radix paeoniae alba:the safflower:the Rhizoma dioscoreae:the angelica sinensis:the L-carnitine:the sodium bicarbonate is 6:15:15:20:20:10:20:10:4:6.

18. The multi-vitamin for adjuvant treatment of AIDS as claimed in claim 15, wherein a weight ratio of the shiitake peptide:the Cordyceps sinensis:the Dendrobium officinale:the prepared rhubarb:the Radix paeoniae alba:the safflower:the Rhizoma dioscoreae:the angelica sinensis:the L-carnitine:the sodium bicarbonate is 4:5:5:10:10:5:10:5:2:3.

19. The multi-vitamin for adjuvant treatment of AIDS as claimed in claim 15, wherein a weight ratio of the shiitake peptide:the Cordyceps sinensis:the Dendrobium officinale:the prepared rhubarb:the Radix paeoniae alba:the safflower:the Rhizoma dioscoreae:the angelica sinensis:the L-carnitine:the sodium bicarbonate is 6:12:13:16:15:5:15:6:4:3.

20. The multi-vitamin for adjuvant treatment of AIDS as claimed in claim 15, wherein a weight ratio of the shiitake peptide:the Cordyceps sinensis:the Dendrobium officinale:the prepared rhubarb:the Radix paeoniae alba:the safflower:the Rhizoma dioscoreae:the angelica sinensis:the L-carnitine:the sodium bicarbonate is 4:10:10:18:18:10:10:5:3:5.
